(19) United States
(12) Patent Application Publication
(10) Pub. No.: US 2004/0133262 A1
(43) Pub. Date: Jul. 8, 2004

(54) Vipul's forward force balloon with firm grip cocktail stent

(76) Inventor: Vipul Narain Roy, New Delhi (IN)

Correspondence Address: DR VIPUL NARAIN ROY, C/O DR ATUL ROY, 18154 TRUFLE LANE, BOYDS, MD, 20841, US

(21) Appl. No.: 10/628,207

(22) Filed: Jul. 29, 2003

Related U.S. Application Data
(60) Provisional application No. 60/399,396, filed on Jul. 31, 2002.

(51) Int. Cl. A61F 2/06

(52) U.S. Cl. 623/1.11; 623/1.42

(57) ABSTRACT

This balloon catheter will have a special shaft having spring like structure which will help in transmitting the force to distal end and thus helping to cross the tight lesions. The balloon material with help of heatexpansile material will strongly grip the undeployed stent and prevent dislodgment of undeployed stent. The cocktail of drug to prevent acute/sub-acute closure and restenosis.

Complete specification for pursuance of patent Application No. 60/399,396 dated 31 Jul. 2002 filed as provisional application

### CROSS REFERENCE TO RELATED APPLICATIONS

Application No. 60/399,396 dated 31 Jul. 2002

### FEDERALLY SPONSORED RESEARCH OR DEVELOPMENT

Not applicable

### REFERENCE TO SEQUENCE LISTING, A TABLE, OR A COMPUTER PROGRAM LISTING COMPACT DISK APPENDIX

Not applicable

### BACKGROUND TO INVENTION

Problem

The standard balloon catheter (coronary and peripheral) and mounted/bare stent has following limitations

If there is obstruction in its path then further push will initially push the guiding catheter back and in next step the guidewire-balloon assembly will start curving down and finally the guidewire with balloon will fall back.

The stent occasionally comes off balloon and embolises some where in vascular bed.

Acute/Subacute Obstruction.

Restenosis.

During expansion while there is further reduction in the length of catheter there is further shear injury to vascular wall.

### BRIEF SUMMARY OF INVENTION

In the balloon proximal to balloon the shaft will be missing in a significant length and this part will have a spring

The stent segment will be short in length, zigzag pattern for expansion and prevent shortening, thin stent, flat outer surface, larger zigzag segment window to allow side branch access.

The outer surface of balloon is such that it has small amount of heat expansible material

The stent will have multiple drug coating

### BRIEF DESCRIPTION OF SEVERAL VIEWS OF DRAWING

FIG. 1

It explains the two types of springs incorporated in the shaft

FIG. 2

It explains the relationship of heat expansile material on balloon and stent struts

(C) Flat inner and outer surface of struts (struts to be thin)

(D) Heatexpansile material on balloon

(E) Compressive outer surface not allowing heatexpansile material to come on outer surface of stent to trap it, it also does not allow increase in stent profile

(F) Balloon inner surface such that the heatexpansile material can not come and disturb the lumen of the balloon

### DETAILED DESCRIPTION OF THE INVENTION

The standard balloon catheter (coronary and peripheral) and mounted/bare stent has following limitations

If there is obstruction in its path then further push will initially push the guiding catheter back and in next step the guidewire-balloon assembly will start curving down and finally the guidewire with balloon will fall back.

The stent occasionally comes off balloon and embolises some where in vascular bed.

Acute/Subacute Obstruction.

Restenosis.

During expansion while there is further reduction in the length of catheter there is further shear injury to vascular wall.

Following modifications will help

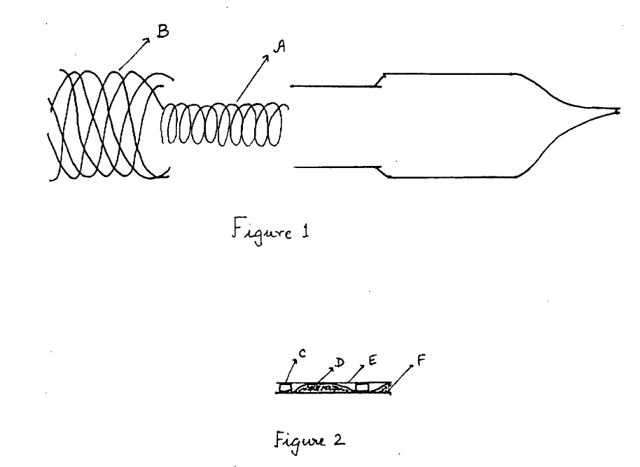

Forward force; In the balloon proximal to balloon the shaft will be missing in a significant length and this part will have a spring (FIG. 1A), it will slowly get compressed in length with the force and transmit it forward. This segment will also have spirally running wires (FIG. 1B) to act like a spring and absorb the force to transmit it forward.

Above segment will be in part of balloon where guide wire is inside the shaft and above modification so that the direction is not lost.

The balloon will be supplied in a fashion that the above segment is straight and the wire does not go through the gap of spring.

Alternatively a wire supplied with a receptacle at the distal segment to allow attaching to the guidewire.

It will be in two types of strength, for proximal lesions (the target lesion being close to guiding catheter tip as proximal LAD) and distal lesions (the target lesion being distant to guiding catheter tip as distal RCA)

The stent segment will be short in length, zigzag pattern for expansion and prevent shortening, thin stent, flat outer surface, larger zigzag segment window to allow side branch access.

The outer surface of balloon is such that it has small amount of heat expansible material to match the window on stent. The compressed balloon is heated and this material fits in this window (thus firmly grips the stent when not deployed). The flat outer surface of stent (FIG. 2C) is in contact with the compressing surface so the heat-expanded material does not come over it and entraps the stent.

The expansible material does not come towards inside of balloon so as to affect its function.

The expansible material does not binds with the stent material.

The expansible material cannot come off the outer surface of the balloon.

Since the expansible material is inside the compressing surface it matches the outer diameter of stent without increasing the profile of mounted stent (FIG. 2D).

When balloon is expanded the window enlarges and stent is free from expansible material.

The stent is coated with a cocktail of following drugs

a. Heparin (unfractionated/low molecular); It will be released over 10 days and maximal in first day and later slowly to prevent acute/subacute obstruction.

b. IIbIIIa receptor blocker (as abciximab); It will be released over 10 days and maximal in first day and later slowly to prevent acute/subacute obstruction.

c. A cytostasis/cytotoxic agent to be released from 14-45 days depending upon agent and dose to prevent restenosis.

Similar peripheral balloon with/without stent

This system will ensure ease in performing difficult cases including the direct stenting. This will also help as less expected acute/subacute obstruction and restenosis incidence

Note

Once the spring force is transmitted the forward movement may have a jump depending upon the compression on the spring. We have seen in rotablator use that such jump has no clinical consequence.

The expandable material to cover some selected segments and not in whole circumference as it will cause problem in deployment.

Balloon to have a longer flexible nose (05-20 mms)

Stent is short segments

More drug at the proximal segment of the stent

What I claim as my invention is:

1 Vipul's forward force balloon with firm grip cocktail stent having spring/spring like structure in its shaft (for transmitting force and maintaining the alignment)
Balloon will have heatexpansile material and longer nose cone
Zig zag pattern stent with a cocktail of drug coating

2 Forward force balloon with firm grip cocktail stent as claimed in claim 1 wherein the balloon catheter shaft will have spring/spring like structure (metallic or non metallic)

3 Forward force balloon with firm grip cocktail stent as claimed in claim 1 wherein spring has circular spirals/strands

4 Forward force balloon with firm grip cocktail stent as claimed in claim 1 wherein spring has longitudinal strands/spirals

5 Forward force balloon with firm grip cocktail stent as claimed in claim 1 wherein spring has oblique strands/spirals

6 Forward force balloon with firm grip cocktail stent as claimed in claim 1 wherein spring/spring like structure will be in portion with or without the PTCA guide wire

7 Forward force balloon with firm grip cocktail stent as claimed in claim 1 wherein the segment is straight and PTCA guide wire will not come out of the gaps of spring like structure

8 Forward force balloon with firm grip cocktail stent as claimed in claim 1 will have spring like structure, will have various lengths and various placements in the shaft

9 Forward force balloon with firm grip cocktail stent as claimed will have stent structure in zig-zag pattern

10 Forward force balloon with firm grip cocktail stent as claimed in claim 9 wherein stent struts will have flat outer surface

11 Forward force balloon with firm grip cocktail stent as claimed in claim 9 wherein zig zag pattern segment length will be short 1-4 mm

12 Forward force balloon with firm grip cocktail stent as claimed will have a cocktail of heparin (unfractionated/fractionated), IIb/IIIa receptor blocker and a cytostasis agents

13 Forward force balloon with firm grip cocktail stent as claimed in claim 12 will have differential release pattern of different drugs

14 Forward force balloon with firm grip cocktail stent as claimed in claim 12 will have more drugs at the edges of the stent and especially the proximal end

15 Forward force balloon with firm grip cocktail stent will have a balloon with heatexpansile material on outer surface in some parts or whole and longer flexible nose cone (02-20 mm)

16 Forward force balloon with firm grip cocktail stent as claimed in claim 15 wherein the heatexpansile material will not affect the lumen of balloon

17 Forward force balloon with firm grip cocktail stent as claimed in claim 15 wherein the heatexpansile material will not bind with the stent

18 Forward force balloon with firm grip cocktail stent as claimed in claim 15 wherein the heatexpansile material will not come off the balloon.

19 Vipul's forward force balloon with firm grip cocktail stent substantially as herein described and illustrated in the accompanying drawings.
Dated this 29^{th} day of July, 2003
